(19) United States
(12) Patent Application Publication
(10) Pub. No.: US 2022/0064436 A1
(43) Pub. Date: Mar. 3, 2022

(54) Gradient Injection Comprising a Mixture of Polymer Microspheres

(71) Applicant: Hangzhou Singclean Medical Products Co., Ltd, Hangzhou (CN)

(72) Inventors: Jie Li, Hangzhou (CN); Feifei Wu, Hangzhou (CN); Kui Zhou, Hangzhou (CN); Yang Fu, Hangzhou (CN); Shitu Ma, Hangzhou (CN)

(21) Appl. No.: 17/197,022

(22) Filed: Mar. 9, 2021

(30) Foreign Application Priority Data
Aug. 27, 2020 (CN) 202010876772.7

(51) Int. Cl. C08L 67/04

(52) U.S. Cl. CPC C08L 67/04

(57) ABSTRACT

Provided is a gradient injection containing a mixture of polymer microspheres. The injection is a freeze-dried powder composed of a filler, cross-linked sodium hyaluronate microspheres, poly-L-lactic acid (PLLA) microspheres, and an excipient. The powder can be mixed with sterile water and then used for injection cosmetology. A triple gradient effectiveness is provided by mixing the filler, the cross-linked sodium hyaluronate microspheres, and the PLLA microspheres to improve the subcutaneous capacity of human skin and repair skin wrinkles, folds, scars, and aging. The gradient injection is also suitable for repairing large-volume facial fat loss without adverse reactions such as nodules and granulomas.

### CROSS REFERENCE TO RELATED APPLICATIONS

The present application claims the benefit of Chinese Patent Application No. 202010876772.7 filed on Aug. 27, 2020, the contents of which are incorporated herein by reference in their entirety.

### FIELD

The present disclosure relates to a gradient injection comprising a mixture of polymer microspheres, and belongs to the technical field of medical cosmetic materials.

### BACKGROUND

Human skin contains a lot of collagen. The growth, repair, nutrition, elasticity, tension and so on of the skin are all closely related to collagen. The aging process of human skin, to a large extent, is a process of collagen losing. Specifically, the human body begins to lose collagen from the age of 25. The loss of collagen is also the main reason why the elderly has skin with reduced thickness and elasticity, as well as wrinkles and fine lines. Therefore, a fundamental method of repairing wrinkles and folds on human skin is to replace the lost collagen.

U.S. Pat. No. 6,716,251B1 discloses an injection implant. Its product (trade name: Sculptra) has been used for dermal and subcutaneous injections to improve wrinkles and facial depression. The main components of the product are degradable poly-L-lactic acid (PLLA) microspheres, and the basic mechanism thereof is as follows. The degradation mechanism of the microspheres is hydrolysis and cell phagocytosis, and the degradation process is slow. During the degradation process, a large number of macrophages and other immune cells are attracted by stimulating the host's immune response, which results in the proliferation of fibroblasts and the differentiation of myofibroblasts, as well as synthesis of a large amount of collagen, thereby restoring the subcutaneous capacity and making up for the loss of collagen (Facial Plast. Surg., 2009, 25:95-99). This product has good effects and a long action time (up to 3 years), and is more advantageous than pure injection fillers such as sodium hyaluronate gel and collagen gel. However, PLLA microspheres have a gradual stimulation and induction effect and cannot produce an effect immediately after being injected. Furthermore, PLLA is a hydrophobic polyester material, and subcutaneous injection with high levels of PLLA can easily cause adverse reactions such as nodules and granulomas. Plast. Reconstr. Surg., 2006, 118(3S): 46S-54S reported that the incidence of adverse reactions after injection with Sculptra was up to 1.2%, and pointed out that the high concentration of microspheres and aggregations thereof are main causes of adverse reactions. PLLA microspheres are the main active ingredients of Sculptra, and its content has been verified by clinical trials. The microspheres at a reduced concentration however are not enough to effectively stimulate regeneration of collagen in skin. Therefore, although the product has good effectiveness, it has problem in safety.

Sodium hyaluronate is also a common material used to repair skin wrinkles. At present, there are many commercially available injection products using HA as the raw material. The main component of these products is a homogeneous gel or a suspension of gel particles formed from chemically crosslinked or uncrosslinked HA and water. The action mechanism of such products is subcutaneous filling only, and therefore their effects in maintaining repaired wrinkles are short (usually only 6 to 18 months). Published Chinese patent application CN109224127A discloses a method for preparing sodium hyaluronate microspheres. Due to their high crosslinking, high uniformity and good mechanical strength, the prepared microspheres have similar effects as PLLA microspheres have. That is, these microspheres can easily stimulate immune response in the body, thereby inducing the regeneration of collagen. In addition, HA microspheres are highly hydrophilic and thus cannot easily cause adverse reactions such as nodules in the human body. However, HA micro spheres are easily degraded by enzymes in the human body, resulting in a relatively short lasting effect of injected and filled products. Therefore, although the use of HA microspheres alone exhibits relatively good safety, it can hardly achieve excellent effectiveness similar to those produced by Sculptra.

### SUMMARY

In view of the above-mentioned shortcomings in the safety and effectiveness of the existing products, the present disclosure aims to provide a gradient injection comprising a mixture of polymer microspheres, which has quick and long-lasting effects.

In order to solve the technical problem(s), the following technical solutions are provided by the present disclosure.

A gradient injection comprising a mixture of polymer microspheres, characterized in that, the injection comprises a filler, cross-linked sodium hyaluronate microspheres, PLLA microspheres, and an excipient. According to the gradient injection of the present disclosure, three effective ingredients—the filler, the cross-linked sodium hyaluronate microspheres, and the PLLA microspheres are mixed to provide a triple gradient effectiveness. The filler can play a subcutaneous filling role at an initial stage of injection. However, the filler is easily degraded and absorbed by tissues and such filling maintains only 1-3 months. This is the first gradient. As the filler is degraded, both the PLLA microspheres and the cross-linked sodium hyaluronate microspheres, as the active ingredients, stimulate and induce regeneration of a large amount of subcutaneous collagen, which restores skin capacity. This stage can last for up to 10-14 months and is the second gradient. After the cross-linked sodium hyaluronate microspheres are degraded, the remaining PLLA microspheres can continuously provide effects of stimulation and induction, which maintains regeneration of collagen at a certain amount. The regenerated collagen balances the naturally lost collagen, which maintains the collagen capacity regenerated in the second gradient, making the injection effect last for 30-36 months until the microspheres are completely degraded and lose effectiveness. This stage is the third gradient. In addition, the cross-linked sodium hyaluronate microspheres can dilute and disperse the hydrophobic PLLA microspheres, and reduce the dosage of the PLLA microspheres, avoiding adverse reactions such as nodules and granulomas caused by the large-dosage use and aggregation of the PLLA microspheres, by way of which product safety is ensured.

According to the present disclosure, a method of preparing the above-mentioned filler is provided. The method is simple to operate and easy to implement.

The method of the present disclosure is as follows.

(1) Preparing PLLA microspheres: dissolving PLLA in dichloromethane to prepare a polymer solution with a mass concentration of 0.5-12%, which is named oil phase 1; weighing and adding a water-soluble surfactant into water, followed by stirring and dissolving to obtain an aqueous solution with a mass concentration of 0.5-5%, which is named aqueous phase 1; adding oil phase 1 into aqueous phase 1 (the oil-water ratio is 1:2-25), followed by stirring and emulsifying for 10-120 minutes to obtain an oil-in-water emulsion; continuously stirring the emulsion at 20-40° C. to cause dichloromethane to completely volatilize, followed by centrifugation to obtain PLLA microspheres; washing the PLLA microspheres with water and ethanol separately, followed by drying in vacuum to obtain powdery solid PLLA microspheres; and screening the powdery solid PLLA microspheres to obtain microspheres with a particle size in a desired range.

(2) Preparing cross-linked sodium hyaluronate microspheres: dissolving sodium hyaluronate powder in water containing 1% sodium hydroxide to obtain a sodium hyaluronate solution with a mass concentration of 5%-30%, which is named aqueous phase 2; weighing and adding an oil-soluble surfactant into light liquid paraffin, followed by stirring to obtain a solution with a mass concentration of 0.5-5%, which is named oil phase 2; adding aqueous phase 2 into oil phase 2 (the oil-water ratio is 3-18:1), followed by stirring and emulsifying for 5-100 minutes to obtain a water-in-oil emulsion; adding, under stirring, divinyl sulfone as a crosslinking agent that is 0.2%-10% of a mass of the sodium hyaluronate powder into the emulsion, followed by stirring at room temperature for 1-10 hours to complete a cross-linking reaction to form cross-linked sodium hyaluronate microspheres; washing the cross-linked sodium hyaluronate microspheres with ethanol, n-hexane, and ethyl acetate separately, followed by drying in vacuum to obtain powdery solid cross-linked sodium hyaluronate microspheres; and screening the powdery solid cross-linked sodium hyaluronate microspheres to obtain microspheres with a particle size in a desired range.

(3) Preparing a gradient injection: adding the obtained PLLA microspheres and the obtained cross-linked sodium hyaluronate microspheres in a certain mass ratio into water, followed by stirring to uniformly disperse the two, and adding the filler that is 30%-150% of a total mass of the microspheres and the excipient that is 70%-150% of the total mass of the microspheres, followed by well stirring to obtain a uniform solution; and freeze-drying the solution to obtain the injection.

Further, a molecular weight of the PLLA in step (1) is 40-300 kDa. Microspheres with a particle size of 3-85 μm, preferably 20-63 μm can be obtained by screening the PLLA microspheres prepared according to step (1).

A molecular weight of the sodium hyaluronate in step (2) is 100-2000 kDa. Microspheres with a particle size of 3-85 μm, preferably 10-45 μm can be obtained by screening the cross-linked sodium hyaluronate microspheres prepared according to step (2).

In the present disclosure, the filler is a common injection material. The filler comprises one or more of sodium hyaluronate, collagen, and sodium carboxymethyl cellulose.

In the present disclosure, the excipient comprises one or more of mannitol, dextran, and glucose. The excipient is used for freeze-drying and forming of products.

In the present disclosure, a mass ratio of the PLLA microspheres to the cross-linked sodium hyaluronate microspheres is (2-88):(98-12), preferably (25-62):(75-38).

In the present disclosure, the filler is 30%-150% of a total mass of the PLLA microspheres and the cross-linked sodium hyaluronate microspheres.

Furthermore, the water-soluble surfactant in step (1) includes but is not limited to one or more of Tween 20, Tween 60, gelatin, and polyvinyl alcohol. The water-soluble surfactant is for emulsifying oil phase 1 and aqueous phase 1 to obtain the oil-in-water emulsion.

The oil-soluble surfactant in step (2) includes but is not limited to one or more of Span 20, Span 60, and Span 80. The oil-soluble surfactant is for emulsifying oil phase 2 and aqueous phase 2 to obtain the water-in-oil emulsion.

In the technical solutions according to the present disclosure, both the PLLA microspheres and the cross-linked sodium hyaluronate microspheres can be substituted with materials having similar effects. For example, the PLLA microspheres can be substituted with microspheres prepared by polyglycolic acid-lactic acid copolymer (PLGA), poly-p-dioxanone (PPDO), polytrimethylene carbonate (PTMC) and other similar polyester materials instead of PLLA. The cross-linked sodium hyaluronate microspheres can be substituted with microspheres prepared by naturally absorbable materials such as collagen, chitosan, starch, gelatin or cellulose instead of cross-linked sodium hyaluronate. The mass ratio of these components can also refer to the above mass ratio. The above alternatives are extensions made at current level technology.

The gradient injection comprising a mixture of polymer microspheres provided by the present disclosure is in the form of a freeze-dried powder, which can be fully mixed with sterile water and then used for medical cosmetology. The gradient injection can improve the subcutaneous capacity of human skin and repair skin wrinkles, folds, scars, and aging. The gradient injection is also suitable for repairing large-volume facial fat loss.

The present disclosure has the following advantages.

(1) The present disclosure combines the advantages of the existing technical solutions and intelligently mixes the three effective ingredients—the filler, the cross-linked sodium hyaluronate microspheres, and the PLLA microspheres, which can provide a triple gradient effectiveness in the skin. It takes effect immediately after the injection, and the cosmetic effect lasts for a relatively long period of 30-36 months.

(2) A mixture of two kinds of microspheres are used in the present disclosure, and the hydrophobic PLLA microspheres are diluted and dispersed, which reduces the dosage of the PLLA microspheres, avoiding adverse reactions such as nodules and granulomas, by way of which product safety is ensured.

### DETAILED DESCRIPTION OF THE EMBODIMENTS

The embodiments of the present disclosure are described below to further clarify the essential features and significant progress of the present disclosure. However, the present disclosure is by no means limited to the embodiments.

### Example 1

(1) Preparing PLLA microspheres: 6 g of PLLA (having an average molecular weight of 156 kDa) was dissolved in 114 g of dichloromethane to prepare a polymer solution with a mass concentration of 5%, which was named oil phase 1. 10 g of Tween 20 was weighed and added into 490 g of water, followed by stirring and dissolving to obtain an aqueous solution with a mass concentration of 2%, which was named aqueous phase 1. All of oil phase 1 was added into aqueous phase 1, followed by stirring and emulsifying for 30 minutes to obtain an oil-in-water emulsion. The emulsion was continuously stirred at 30° C. for 12 hours to cause dichloromethane to completely volatilize, and then centrifuged to obtain PLLA microspheres. The PLLA microspheres were washed with water and ethanol separately, followed by drying in vacuum, obtaining 5.6 g of powdery solid PLLA microspheres. The microspheres were then screened with a 63-μm sieve and a 20-μm sieve respectively, obtaining 4.1 g of PLLA microspheres with a particle size of 20-63 μm.

(2) Preparing cross-linked sodium hyaluronate microspheres: 6 g of sodium hyaluronate powder (having an average molecular weight of 960 kDa) was dissolved in 44 g of water containing 1% sodium hydroxide to prepare a sodium hyaluronate solution with a mass concentration of 12%, which was named aqueous phase 2. 6 g of Span 80 was weighed and added into 294 g of light liquid paraffin, followed by stirring to obtain a solution with a mass concentration of 2%, which was named oil phase 2. All of aqueous phase 2 was added into oil phase 2, followed by stirring and emulsifying for 20 minutes to obtain a water-in-oil emulsion. Divinyl sulfone as a crosslinking agent that was 1.5% of the mass of the sodium hyaluronate powder was added into the emulsion under stirring, followed by stirring at room temperature for 4 hours to complete a cross-linking reaction to form cross-linked sodium hyaluronate microspheres. The cross-linked sodium hyaluronate microspheres were washed with ethanol, n-hexane, and ethyl acetate separately, followed by drying in vacuum, obtaining 5.1 g of powdery solid cross-linked sodium hyaluronate microspheres. The microspheres were screened with a 45-μm sieve and a 10-μm sieve respectively, obtaining 4.6 g of cross-linked sodium hyaluronate microspheres with a particle size of 10-45 μm.

(3) Preparing a gradient injection: 4.1 g of the prepared PLLA microspheres and 4.6 g of the prepared cross-linked sodium hyaluronate microspheres were added into 300 mL of water, followed by stirring to uniformly disperse the two. Then 1.7 g of sodium hyaluronate powder (having an average molecular weight of 1750 kDa) and 4.5 g of mannitol were added respectively, followed by well stirring to obtain a uniform solution. 5 mL of the solution was placed into a 7-mL vial and freeze-dried to obtain an injection.

### Example 2

(1) Preparing PLLA microspheres: 300 g of an oil phase solution containing 0.5% PLLA (having an average molecular weight of 260 kDa) in dichloromethane was prepared according to the same method as used in Example 1. 2000 g of an aqueous phase solution containing 5% Tween 60 was prepared. The two phases were mixed and stirred for 10 minutes, followed by continuous stirring at 40° C. for 12 hours to cause dichloromethane to completely volatilize, and then centrifuged to obtain PLLA microspheres. The PLLA microspheres were washed and dried in vacuum, obtaining 1.4 g of PLLA microspheres. The microspheres were then screened with a 85-μm sieve and a 40-μm sieve respectively, obtaining 0.9 g of PLLA microspheres with a particle size of 40-85 μm.

(2) Preparing cross-linked sodium hyaluronate microspheres: 100 g of an aqueous solution containing 5% sodium hyaluronate (having an average molecular weight of 1750 kDa) was prepared according to the same method as used in Example 1. 600 g of an oil phase solution containing 5% Span 80 in liquid paraffin was prepared. The two phases were mixed and stirred for 5 minutes to form an emulsion. Divinyl sulfone that was 0.2% of the mass of the sodium hyaluronate powder was added into the emulsion, followed by stirring at room temperature for 10 hours to complete a cross-linking reaction to form cross-linked sodium hyaluronate microspheres. The cross-linked sodium hyaluronate microspheres were washed with ethanol, n-hexane, and ethyl acetate separately, followed by drying in vacuum, obtaining 4.8 g of powdery solid cross-linked sodium hyaluronate microspheres. The microspheres were then screened with a 85-μm sieve and a 40-μm sieve respectively, obtaining 3.6 g of cross-linked sodium hyaluronate microspheres with a particle size of 40-85 μm.

(3) Preparing a gradient injection: The prepared PLLA microspheres, the prepared cross-linked sodium hyaluronate microspheres, 2.5 g of collagen, and 5.0 g of dextran were mixed in 200 mL of water according to the same method as used in Example 1 to prepare a uniform solution. 5 mL of the solution was placed into a 7-mL vial and freeze-dried to obtain an injection.

### Example 3

(1) Preparing PLLA microspheres: 50 g of an oil phase solution containing 12% PLLA (having an average molecular weight of 52 kDa) was prepared according to the same method as used in Example 1. 1250 g of an aqueous phase solution containing 0.5% sodium lauryl sulfate was prepared. The two phases were mixed and stirred for 120 minutes, followed by continuous stirring at 25° C. for 12 hours to cause dichloromethane to completely volatilize, and then centrifuged, to obtain PLLA microspheres. The PLLA microspheres were washed and dried in vacuum, obtaining 5.8 g of PLLA microspheres. The microspheres were then screened with a 25-μm sieve and a 3-μm sieve respectively, obtaining 3.2 g of PLLA microspheres with a particle size of 3-25 μm.

(2) Preparing cross-linked sodium hyaluronate microspheres: 10 g of an aqueous solution containing 30% sodium hyaluronate (having an average molecular weight of 130 kDa) was prepared according to the same method as used in Example 1. 180 g of an oil phase solution containing 1% Span 20 and 0.5% Span 80 in liquid paraffin was prepared. The two phases were mixed and stirred for 100 minutes to form an emulsion. Divinyl sulfone that was 10% of the mass of the sodium hyaluronate powder was added into the emulsion, followed by stirring at room temperature for 1 hour to complete a cross-linking reaction to form cross-linked sodium hyaluronate microspheres. The cross-linked sodium hyaluronate microspheres were washed with ethanol, n-hexane, and ethyl acetate separately, followed by drying in vacuum, obtaining 2.9 g of powdery solid cross-linked sodium hyaluronate microspheres. The microspheres were then screened with a 3-μm sieve and a 25-μm sieve respectively, obtaining 1.3 g of cross-linked sodium hyaluronate microspheres with a particle size of 3-25 μm.

(3) Preparing a gradient injection: The prepared PLLA microspheres, the prepared cross-linked sodium hyaluronate microspheres, 6.8 g of sodium carboxymethyl cellulose, and 6 g of glucose were mixed in 200 mL of water according to the same method as used in Example 1 to prepare a uniform solution. 5 mL of the solution was placed into a 7-mL vial and freeze-dried to obtain an injection.

Table 1 is a list of test results of the products prepared in Examples 1-3.

TABLE 1

| Requirements for | Example | | Example | | Example | |
|---|---|---|---|---|---|---|
| Performance | 1 | | 2 | | 3 | |
| Appearance: white | met | | met | | met | |
| flocculent powder | | | | | | |
| Solubility: After adding | met | | met | | met | |
| 5 mL of sterile water and | | | | | | |
| shaking for 1-3 minutes, | | | | | | |
| it should be a uniform | | | | | | |
| suspension without | | | | | | |
| visible flocculent or | | | | | | |
| lumpy precipitation. | | | | | | |
| Pushing force of Solution | 24N | | 32N | | 20N | |
| (26 G needle): ≤35N | | | | | | |
| pH of solution: 5.5-8 | 5.8 | | 6.3 | | 5.9 | |
| Heavy metals in solution: | ≤10 | μg/g | ≤10 | μg/g | ≤10 | μg/g |
| ≤10 μg/g | | | | | | |
| Dichloromethane residue: | not | | not | | not | |
| ≤0.5% | detected | | detected | | detected | |
| Divinyl sulfone residue: | 1.1 | μg/g | 0.8 | μg/g | 2.5 | μg/g |
| ≤3 μg/g | | | | | | |

| | | | |
|---|---|---|---|
| Cytotoxicity: not greater | met | met | met |
| than level 1 | | | |
| Skin sensitization: no | met | met | met |
| sensitization | | | |
| Intradermal irritation: no | met | met | met |
| intradermal irritation | | | |

The present disclosure is not limited to the above-mentioned embodiments. If there are any changes or modifications to the present disclosure that do not depart from the spirit and scope of the present disclosure and these changes and modifications fall within scopes of technical solutions equivalent to the claims of the present disclosure, the present disclosure is also intended to include these changes and modifications.

1. A gradient injection comprising a mixture of polymer microspheres, wherein the gradient injection comprises a filler, cross-linked sodium hyaluronate microspheres, poly-L-lactic acid PLLA microspheres, and an excipient.

2. The gradient injection according to claim 1, wherein the filler, the cross-linked sodium hyaluronate microspheres, and the PLLA microspheres form a triple gradient system, wherein a first gradient of the gradient system is provided by the filler, a second gradient thereof is provided by both the cross-linked sodium hyaluronate microspheres and the PLLA microspheres, and a third gradient thereof is provided by the PLLA microspheres.

3. The gradient injection according to claim 1, wherein the gradient injection is prepared by the following method:
(1) preparing PLLA microspheres: dissolving PLLA in dichloromethane to prepare a polymer solution with a mass concentration of 0.5-12%, which is named oil phase 1; weighing and adding a water-soluble surfactant into water, followed by stirring and dissolving to obtain an aqueous solution with a mass concentration of 0.5-5%, which is named aqueous phase 1; adding oil phase 1 into aqueous phase 1 in an oil-water ratio of 1:2-25, followed by stirring and emulsifying for 10-120 minutes to obtain an oil-in-water emulsion; continuously stirring the emulsion at 20-40° C. to cause dichloromethane to completely volatilize, followed by centrifugation to obtain PLLA microspheres; washing the PLLA microspheres with water and ethanol separately, followed by drying in vacuum to obtain powdery solid PLLA microspheres; and screening the powdery solid PLLA microspheres to obtain microspheres with a particle size in a desired range;
(2) preparing cross-linked sodium hyaluronate microspheres: dissolving sodium hyaluronate powder in water containing 1% sodium hydroxide to obtain a sodium hyaluronate solution with a mass concentration of 5%-30%, which is named aqueous phase 2; weighing and adding an oil-soluble surfactant into light liquid paraffin, followed by stirring to obtain a solution with a mass concentration of 0.5-5%, which is named oil phase 2; adding aqueous phase 2 into oil phase 2 in an oil-water ratio of 3-18:1, followed by stirring and emulsifying for 5-100 minutes to obtain a water-in-oil emulsion; adding, under stirring, divinyl sulfone as a crosslinking agent that is 0.2%-10% of a mass of the sodium hyaluronate powder into the emulsion, followed by stirring at room temperature for 1-10 hours to complete a cross-linking reaction to form cross-linked sodium hyaluronate microspheres; washing the cross-linked sodium hyaluronate microspheres with ethanol, n-hexane, and ethyl acetate separately, followed by drying in vacuum to obtain powdery solid cross-linked sodium hyaluronate microspheres; and screening the powdery solid cross-linked sodium hyaluronate microspheres to obtain microspheres with a particle size in a desired range.
(3) preparing the gradient injection: adding the obtained PLLA microspheres and the obtained cross-linked sodium hyaluronate microspheres in a certain mass ratio into water, followed by stirring to uniformly disperse the two, and adding the filler that is 6%-110% of a total mass of the microspheres and the excipient that is 70%-150% of the total mass of the microspheres, followed by well stirring to obtain a uniform solution; and freeze-drying the solution to obtain the injection.

4. The gradient injection according to claim 1, wherein
the PLLA microspheres have a particle size of 3-65 μm, and the PLLA has a molecular weight of 40-300 kDa; the crosslinked sodium hyaluronate microspheres have a particle size of 3-65 μm, and the sodium hyaluronate before crossing-linking has a molecular weight of 100-2000 kDa; the filler comprises one or more of sodium hyaluronate, collagen, and sodium carboxymethyl cellulose; and the excipient is one or more of mannitol, dextran, and glucose.

5. The gradient injection according to claim 1, wherein a mass ratio of the PLLA microspheres to the cross-linked sodium hyaluronate microspheres is (2-88):(98-12).

6. A gradient injection comprising a mixture of polymer microspheres, characterized in that, the gradient injection comprises a filler, first microspheres, second microspheres, and an excipient, wherein
the first microspheres are selected from naturally absorbable material microspheres; and
the second microspheres are selected from polyester microspheres.

7. The gradient injection according to claim 6, characterized in that, a mass ratio of the first microspheres to the second microspheres is (2-88):(98-12).

8. The gradient injection according to claim 2, wherein the gradient injection is prepared by the following method:
(1) preparing PLLA microspheres: dissolving PLLA in dichloromethane to prepare a polymer solution with a mass concentration of 0.5-12%, which is named oil phase 1; weighing and adding a water-soluble surfactant into water, followed by stirring and dissolving to obtain an aqueous solution with a mass concentration of 0.5-5%, which is named aqueous phase 1; adding oil phase 1 into aqueous phase 1 in an oil-water ratio of 1:2-25, followed by stirring and emulsifying for 10-120 minutes to obtain an oil-in-water emulsion; continuously stirring the emulsion at 20-40° C. to cause dichloromethane to completely volatilize, followed by centrifugation to obtain PLLA microspheres; washing the PLLA microspheres with water and ethanol separately, followed by drying in vacuum to obtain powdery solid PLLA microspheres; and screening the powdery solid PLLA microspheres to obtain microspheres with a particle size in a desired range;
(2) preparing cross-linked sodium hyaluronate microspheres: dissolving sodium hyaluronate powder in water containing 1% sodium hydroxide to obtain a sodium hyaluronate solution with a mass concentration of 5%-30%, which is named aqueous phase 2; weighing and adding an oil-soluble surfactant into light liquid paraffin, followed by stirring to obtain a solution with a mass concentration of 0.5-5%, which is named oil phase 2; adding aqueous phase 2 into oil phase 2 in an oil-water ratio of 3-18:1, followed by stirring and emulsifying for 5-100 minutes to obtain a water-in-oil emulsion; adding, under stirring, divinyl sulfone as a crosslinking agent that is 0.2%-10% of a mass of the sodium hyaluronate powder into the emulsion, followed by stirring at room temperature for 1-10 hours to complete a cross-linking reaction to form cross-linked sodium hyaluronate microspheres; washing the cross-linked sodium hyaluronate microspheres with ethanol, n-hexane, and ethyl acetate separately, followed by drying in vacuum to obtain powdery solid cross-linked sodium hyaluronate microspheres; and screening the powdery solid cross-linked sodium hyaluronate microspheres to obtain microspheres with a particle size in a desired range.
(3) preparing the gradient injection: adding the obtained PLLA microspheres and the obtained cross-linked sodium hyaluronate microspheres in a certain mass ratio into water, followed by stirring to uniformly disperse the two, and adding the filler that is 6%-110% of a total mass of the microspheres and the excipient that is 70%-150% of the total mass of the microspheres, followed by well stirring to obtain a uniform solution; and freeze-drying the solution to obtain the injection.

9. The gradient injection according to claim 2, wherein
the PLLA microspheres have a particle size of 3-65 μm, and the PLLA has a molecular weight of 40-300 kDa; the crosslinked sodium hyaluronate microspheres have a particle size of 3-65 μm, and the sodium hyaluronate before crossing-linking has a molecular weight of 100-2000 kDa; the filler comprises one or more of sodium hyaluronate, collagen, and sodium carboxymethyl cellulose; and the excipient is one or more of mannitol, dextran, and glucose.

10. The gradient injection according to claim 3, wherein
the PLLA microspheres have a particle size of 3-65 μm, and the PLLA has a molecular weight of 40-300 kDa; the crosslinked sodium hyaluronate microspheres have a particle size of 3-65 μm, and the sodium hyaluronate before crossing-linking has a molecular weight of 100-2000 kDa; the filler comprises one or more of sodium hyaluronate, collagen, and sodium carboxymethyl cellulose; and the excipient is one or more of mannitol, dextran, and glucose.

11. The gradient injection according to claim 2, wherein a mass ratio of the PLLA microspheres to the cross-linked sodium hyaluronate microspheres is (2-88):(98-12).

12. The gradient injection according to claim 3, wherein a mass ratio of the PLLA microspheres to the cross-linked sodium hyaluronate microspheres is (2-88):(98-12).
